(19) United States
(12) Patent Application Publication
(10) Pub. No.: US 2026/0202498 A1
(43) Pub. Date: Jul. 16, 2026

(54) FASTER THREE-DIMENSIONAL MAGNETIC RESONANCE IMAGING

(71) Applicant: The Regents of the University of Colorado, a body corporate, Denver, CO (US)

(72) Inventor: Nicholas DWORK, Denver, CO (US)

(21) Appl. No.: 19/130,920

(22) PCT Filed: Nov. 17, 2023
(86) PCT No.: PCT/US2023/080285

§ 371 Date: May 16, 2025

(51) Int. Cl. G01R 33/56; A61B 5/055

(52) U.S. Cl. CPC G01R 33/5608; A61B 5/055

(57) ABSTRACT

A method to reconstruct an image, from a magnetic resonance imaging (MRI) machine, with reduced sampling is provided. The method includes receiving a first indication corresponding to a support region, obtaining a set of data samples associated with the first indication, determining a subset of data samples, from the set of data samples, that does not overlap with the one or more regions having aliasing, generating a first reconstruction, based on the determined subset of data samples, generating a frequency representation corresponding to the first reconstruction, subtracting the frequency representation from the set of data samples, thereby generating a modified set of data samples, generating a second reconstruction, based on the modified set of data samples, generating a reduced sampling reconstruction corresponding to the support region, by combining the first and second reconstructions, and outputting the reduced sampling reconstruction.

### CROSS-REFERENCE TO RELATED APPLICATION

This application claims priority to U.S. Provisional Application No. 63/426,621, titled “Faster Three-Dimensional Magnetic Resonance Imaging,” filed on Nov. 18, 2022, the entire disclosure of which is hereby incorporated by reference in its entirety.

### BACKGROUND

The utility of a Magnetic Resonance Imaging (MRI) machine is directly related to how long a scan takes. Longer scan times mean the machine is used on fewer patients, may yield images of lower resolution, and may increase motion artifacts that negatively affect scan quality and diagnostic usability. Therefore, it is desirable to reduce scan times for MRI machines for these and a variety of other reasons.

It is with respect to these and other general considerations that embodiments have been described. Also, although relatively specific problems have been discussed, it should be understood that the embodiments should not be limited to solving the specific problems identified in the background.

### SUMMARY

Aspects of the present disclosure relate to methods, systems, and media for performing three-dimensional magnetic resonance (MRI) imaging.

In some examples a method to reconstruct an image, from a magnetic resonance imaging (MRI) machine, with reduced sampling is provided. The method includes receiving a first indication corresponding to a region containing the subject to be imaged (which could be specified as the area within the exterior boundary of a support region) called the field-of-view, and obtaining a set of data samples associated with the first indication. The method further includes determining a subset of data samples, from the set of data samples, generating a first reconstruction, based on the determined subset of data samples, identifying areas of the first reconstruction that are free of aliasing, generating a frequency representation corresponding to the unaliased portions of the first reconstruction, subtracting the frequency representation from the set of data samples, thereby generating a modified set of data samples, generating a second reconstruction, based on the modified set of data samples, generating a reduced sampling reconstruction corresponding to the support region, by combining the first and second reconstructions, and outputting the reduced sampling reconstruction.

In some examples, the field-of-view is generally non-rectangular.

In some examples, the set of data samples are received from one or more sensing coils and a field-of-view is provided for each sensing coil of the one or more sensing coils.

In some examples, the indication corresponding to the field-of-view is generated automatically.

In some examples, the indication corresponding to the field-of-view is generated automatically based on a localizer scan obtained from the MRI machine.

In some examples, the set of data samples are from an under-sampled reconstruction. In the under-sampled reconstruction, aliasing is present across at least a portion of a field-of-view corresponding to the set of data samples.

In some examples, at least a portion of the field-of-view is half of the field-of-view.

In some examples, the set of data samples are obtained from an imaging device.

In some examples, the first and second reconstructions are generated using an inverse two-dimensional discrete Fourier transform (DFT), which may be a fast Fourier transform (FFT).

In some examples, a method to reconstruct an image, from a magnetic resonance imaging (MRI) machine, with reduced sampling is provided. The method includes receiving an indication corresponding to a field-of-relevance, generating a sampling interval, based on the received indication, causing the MRI machine to collect data samples at the sampling interval, and generating a reconstruction, based on the data samples collected for the sampling interval. The reconstruction has aliasing resulting from the sampling interval.

In some examples, the method further includes determining a region of the reconstruction corresponding to the field-of-relevance, and outputting the region of the reconstruction. The determined region does not have aliasing.

In some examples, the sampling interval comprises a first fixed interval of distances in a first direction at which the data samples are obtained.

In some examples, the sampling interval further comprises a second fixed interval of distances in a second direction at which the data samples are obtained.

In some examples, the sampling interval further comprises additional fixed interval of distances in additional directions at which the data samples are obtained.

In some examples, the first direction is a horizonal direction and the second direction is a vertical direction.

In some examples, the indication corresponding to the field-of-relevance is received via user-input.

In some examples, the indication corresponding to the field-of-relevance is generated automatically.

In some examples, the indication corresponding to the field-of-relevance is generated automatically based on a localizer scan obtained from the MRI machine.

This summary is provided to introduce a selection of concepts in a simplified form that are further described below in the Detailed Description. This summary is not intended to identify key features or essential features of the claimed subject matter, nor is it intended to be used to limit the scope of the claimed subject matter. Additional aspects, features, and/or advantages of examples will be set forth in part in the following description and, in part, will be apparent from the description, or may be learned by practice of the disclosure.

### BRIEF DESCRIPTION OF THE DRAWINGS

Non-limiting and non-exhaustive examples are described with reference to the following Figures.

FIG. 1 illustrates an overview of an example system according to some aspects described herein.

FIG. 2A illustrates a spin warp acquisition that can be modified based on a field-of-relevance, according to some aspects described herein.

FIG. 2B illustrates a sampling pattern that can be modified based on a field-of-relevance, according to some aspects described herein.

FIG. 3 illustrates a simulated magnetic resonance imaging (MRI) reconstruction of a brain, according to some aspects described herein.

FIG. 4 illustrates an example abdominal MRI reconstruction during pregnancy, according to some aspects described herein.

FIG. 5 illustrates an MRI reconstruction of a plurality of components of a body, according to some aspects described herein.

FIG. 6A illustrates an example sampling pattern with a rectangular field-of-view but without taking into account the known support, according to some aspects described herein.

FIG. 6B illustrates an example sampling pattern after taking into account a non-rectangular field-of-view, according to some aspects described herein.

FIG. 7 illustrates a depiction of a flow to reconstruct an image, from fewer samples, with a non-rectangular field-of-view, according to some aspects described herein.

FIG. 8 illustrates a reconstruction of an ankle from fully sampled data and from under-sampled data, according to some aspects described herein.

FIG. 9 illustrates a depiction of generating a full reconstruction, while accounting for a known support, according to some aspects described herein.

FIG. 10 illustrates a plurality of sampling patterns, according to some aspects described herein.

FIG. 11 illustrates an overview of an example method to reconstruct an image, from an MRI machine, with reduced sampling, according to some aspects described herein.

FIG. 12 illustrates an overview of another example method to reconstruct an image, from an MRI machine, with reduced sampling, according to some aspects described herein.

FIG. 13 illustrates a block diagram illustrating example physical components of a computing device with which aspects of the disclosure may be practiced.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Embodiments may be practiced as methods, systems or devices. Accordingly, embodiments may take the form of a hardware implementation, an entirely software implementation, or an implementation combining software and hardware aspects. The following detailed description is therefore not to be taken in a limiting sense, and the scope of the present disclosure is defined by the appended claims and their equivalents.

The utility of a magnetic resonance imaging (MRI) machine may be directly related to how long it takes for the MRI machine to generate a scan. Longer scan times mean that the machine can be used on fewer patients, that images of lower resolution are produced, and that there may be increased motion artifacts (e.g., from movement of the subject being scanned) that negatively affect the quality and diagnostic usability of scans. Several technologies have increased the speed of MRI, such as, for example, partial Fourier sampling, parallel imaging using multiple coils (or sensors) to contemporaneously image different regions of a body, and compressed sensing (e.g., using an assumption of sparsity). In some examples, these methods can be combined with machine learning (e.g., deep learning) to further reduce the number of samples required, for scans, with information gained from past experiences (e.g., previously performed scans). Mechanisms disclosed herein (including systems, methods, and media for reconstructing an image from an MRI machine, with reduced samples) can be combined with existing technologies for relatively fast, high-resolution MRI. Existing technologies with which mechanisms described herein can be combined may be recognized by those of ordinary skill in the art, at least in light of teachings described herein.

In some examples, known information can be incorporated into a scanning and image formation process to reduce the number of data samples that are required to be collected, while retaining a relatively high quality of an image. In some examples, when imaging a patient, a technician can supply a field-of-view that encapsulates a portion of the patient's body to be imaged. In some examples, the field-of-view may be a rectangle. In such instances, the field-of-view may include a region that is absent of anything to image. For example, an axial slice of a brain is oval-shaped, which means that the subject of the MRI (e.g., the brain) will not be present near the corners of the rectangular field-of-view.

In some examples provided herein, a technician supplies a non-rectangular field-of-view (e.g., by selecting from a set of pre-determined shapes and/or by drawing freehand) on routinely acquired localizer images. Teachings described herein are advantageous for altering sampling patterns of MRI to accommodate a non-rectangular field-of-view. Simply altering the sampling pattern to have the shape of the field-of-view may not yield a diagnostic image. Mechanisms provided herein include a method to reduce the number of samples in a pre-determined way that takes advantage of known dead space (e.g., that corresponds to black space in an MRI image).

In some instances, a physician is only interested in a small portion of an imaged patient. For example, the physician may only be interested in observing a heart, a liver, or a fetal brain. Currently, MRI may resolve an entire field-of-view of the patient's body to properly image an embedded organ or other anatomy. Mechanisms provided herein allow a scanning technician to provide an indication (e.g., an additional smaller contour) that corresponds to (e.g., outlines) the relevant structures to be imaged, thereby defining a field-of-relevance. In some examples provided herein, space outside of the field-of-relevance but within the field-of-view is used to eliminate artifacts introduced with reduced sampling.

Some examples provided herein include a system that generates a sampling pattern from a manually specified field-of-relevance and reconstructs an image from those samples. The inputs to the system, or a subcomponent of the system, may include the field-of-view and the field-of-relevance. In some examples, a system is provided that generates a sampling pattern from a non-rectangular field-of-view and reconstructs an image from those samples. In some examples, contours will be selected by hand, such as by a medical professional, e.g., an MRI scanning technologist. In other examples, the contour may be determined, at least in part, automatically. Some examples described herein may be advantageous for demonstrating high quality images, with fewer samples, using knowledge of a rectangular field-of-view and a rectangular field-of-relevance. Additionally, or alternatively, some examples may be advantageous for demonstrating high quality images, with fewer samples, using knowledge of a non-rectangular field-of-view and/or a non-rectangular field-of-relevance. Further, some examples may be advantageous to improve existing technology, in combination with partial Fourier sensing, parallel imaging, and compressed sensing.

In some examples, a field-of-relevance and/or a field-of-view may be provided by a technician from images (e.g., two-dimensional images) routinely acquired at the beginning of a scan, called localizer or scout scans. The images may be quickly acquired in the axial, sagittal, and coronal planes (e.g., one image in each plane). On the images, the technician may select a rectangular field-of-relevance or a non-rectangular field-of-relevance and a rectangular field-of-view or a non-rectangular field-of-view.

In some examples, such as in clinical trials that incorporate mechanisms described herein, a plurality of data sets may be collected, such as 1) fully sampled data, 2) sampling that takes a rectangular field-of-relevance into account, 3) sampling that takes a non-rectangular field-of-relevance into account, 4) sampling that takes a non-rectangular field-of-view into account, and 5) sampling that takes a non-rectangular field-of-view and a non-rectangular field-of-relevance into account. Medical experts (e.g., radiologists) may quantify the quality of the image generated from each set of samples to recognize the advantages of mechanisms described herein. For example, mechanisms described herein can be used to create an MRI scanning process, relatively faster than existing MRI scanning processes, using a non-rectangular field-of-view and a non-rectangular field-of-relevance. Specifically, an MRI scan using mechanisms provided herein may be 10-70% faster than existing MRI scanning technologies, depending on the anatomy imaged. Mechanisms provided herein can empower new applications of MRI including reliable high-resolution fetal imaging and clinical high-resolution dynamic imaging with external contrast.

However, there are confounding factors when using MRI. 1) MRI assumes that there is little motion during the scan. Therefore, cardiac motions, blood flow, respiratory motions, and fetal motions can corrupt the quality of the images. And 2) MRI is inherently slow due to the length of time it takes for an isochromat to polarize with the external magnetic field.

The speed of clinical MRI may be increased with parallel imaging (which uses multiple antennas, rather than one, to simultaneously make multiple measurements), partial Fourier acquisition (which assumes the phase variations across the image are relatively smooth), and compressed sensing (which assumes a sparsity property of the reconstructed image). While these technologies improve the speed of MRIs, a three-dimensional volume may still require about 30 seconds of scan time, which is long enough that motion can significantly degrade the quality of the image. Further, relatively long three-dimensional scan times (e.g., 30 seconds) have limited some MRI applications (e.g., MRI during pregnancy) to two-dimensional imaging, which reduces the amount of information presented to the physician.

To date, the reconstruction of an MR image has been a rectangle. This rectangle is called the field-of-view (FOV) and is specified by the imaging technician prior to scanning. However, the support of MRI (that portion of the image that images the patient rather than space) may be less than the FOV. Much of the FOV lies outside of the body and this is known prior to scanning.

Mechanisms described herein reconstruct a volume from a reduced sampling pattern for a non-rectangular support. Such techniques may be implemented as a stand-alone acceleration method. Additionally, or alternatively, reduced sampling permitted for a non-rectangular support may be combined with additional accelerations of partial Fourier sampling, parallel imaging, and/or compressed sensing, among other examples.

Further, mechanisms described herein provide the ability to automatically estimate a support of an image from (possibly low-resolution) localizing scans. Localizing images take little time and may be acquired by a technician prior to conducting a lengthy diagnostic scan in order to estimate the FOV. The localizing images can then be used to estimate the non-rectangular support of the subject.

Mechanisms described herein may reconstruct a volume from a reduced sampling pattern from a set of non-rectangular supports, such as where there is one non-rectangular support for each image sensor (e.g., sensing coil). In some examples, a set of data samples can be received from one or more image sensors (e.g., sensing coils), such as where a first image sensor of the one or more image sensors is provided a different support region than a second image sensor of the one or more image sensors. In some examples, a set of data samples are received from one or more sensing coils and a field-of-view is provided for each sensing coil of the one or more sensing coils. Such techniques may be implemented as a stand-alone acceleration method. Additionally and/or alternatively, reduced sampling permitted for a set of non-rectangular supports may be combined with additional accelerations of partial Fourier sampling, parallel imaging, and/or compressed sensing, among other examples.

It should be recognized that while some examples disclosed herein may rely on a support or set of supports that is/are estimated by a human (e.g., from localizer scans), other examples provided herein may rely on a support or set of supports that is/are estimated automatically, such as using visual processing, machine-learning, and/or another automatic processing technique that may be recognized by those of ordinary skill in the art. In some examples, the support can be known from sensing coils or anatomy that are used. For example, if one is imaging an ankle with an ankle sensing setup, it may be known ahead of time that the support of the anatomy will look something like an ankle with a whole quarter of the image containing nothing but air. Mechanisms disclosed herein may be applicable for medical use, such as in a clinic or for clinical trials. Additionally, or alternatively, mechanisms disclosed herein may be applicable for veterinary medicine or for non-medical uses, such as preclinical studies (e.g., viewing anatomy of small animals), chemistry (e.g., looking at rocks), etc.

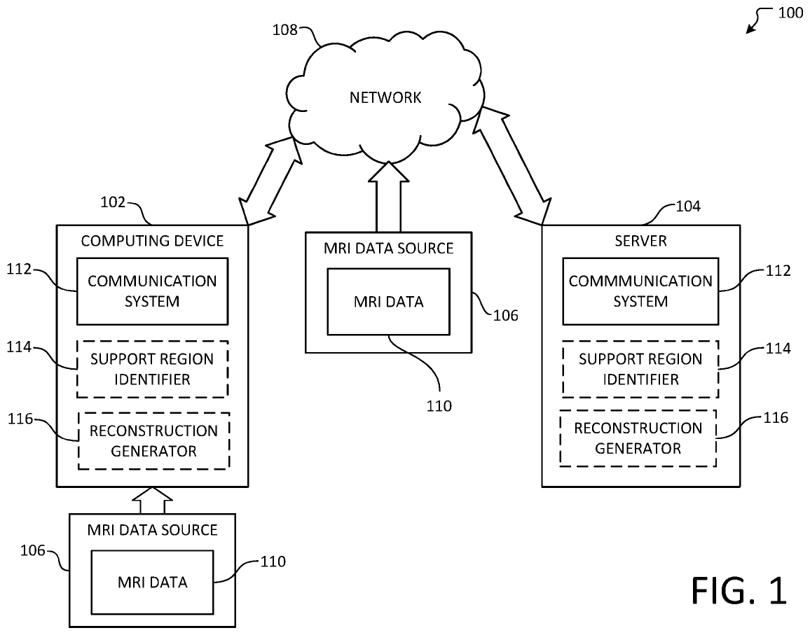

FIG. 1 shows an example of a system 100, in accordance with some aspects of the disclosed subject matter. The system 100 may be a system for reconstructing an image (e.g., from an MRI machine), with reduced sampling. The system 100 includes one or more computing devices 102, one or more servers 104, a magnetic resonance imaging (MRI) data source 106, and a communication network or network 108. The computing device 102 can receive MRI data 110 from the MRI data source 106, which may be, for example, an MRI machine, a database storing MRI data, an application configured to generate MRI data, etc. Additionally, or alternatively, the network 108 can receive MRI data 110 from the MRI data source 106.

Computing device 102 may include a communication system 112, a support region identifier engine or component 114, and/or a reconstruction generator engine or component 116. In some examples, computing device 102 can execute at least a portion of the support region identifier component 114 to identify a support region within an MRI scan, based on the MRI data 110, as will be discussed further herein. Further, in some examples, computing device 102 can execute at least a portion of the reconstruction generator component 116 to generate a reconstruction based on the MRI data 110, or a determined subset thereof, as will be discussed further herein.

Server 104 may include a communication system 112, a support region identifier engine or component 114, and/or a reconstruction generator engine or component 116. In some examples, server 104 can execute at least a portion of the support region identifier component 114 to identify a support region within an MRI scan, based on the MRI data 110, as will be discussed further herein. Further, in some examples, server 104 can execute at least a portion of the reconstruction generator component 116 to generate a reconstruction based on the MRI data 110, or a determined subset thereof, as will be discussed further herein.

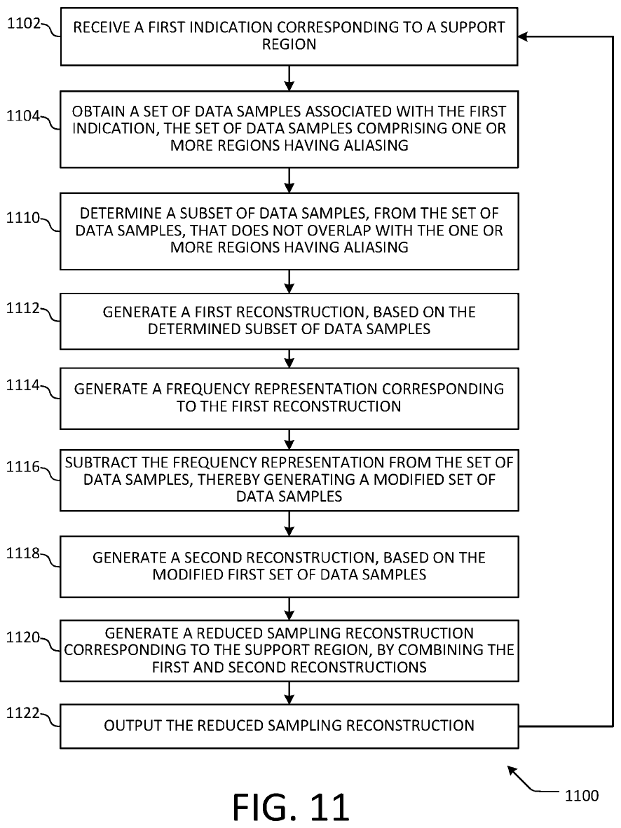

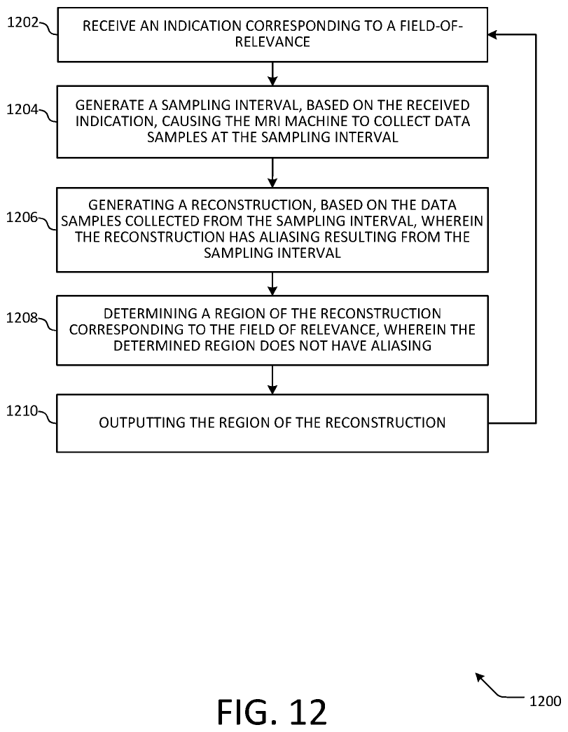

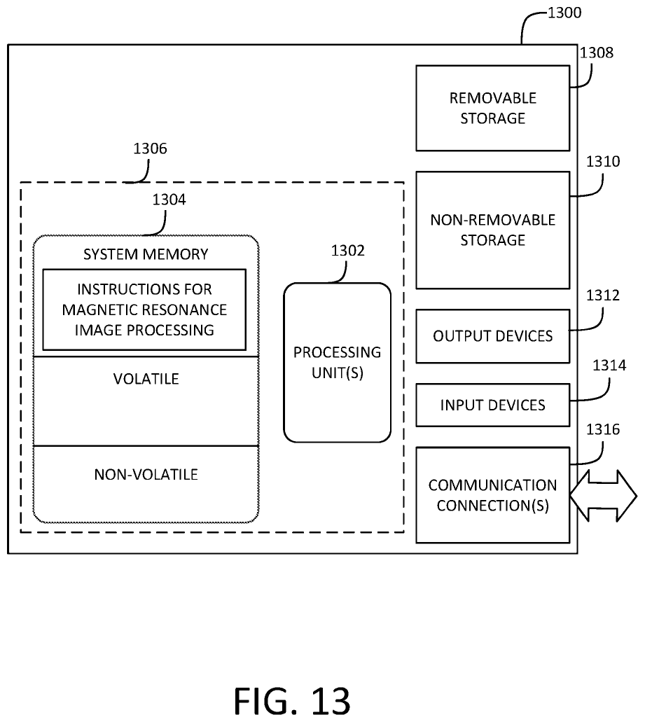

Additionally, or alternatively, in some examples, computing device 102 can communicate data received from MRI data source 106 to the server 104 over a communication network 108, which can execute at least a portion of the support region identifier component 114, and/or the reconstruction generator component 116. In some examples, the support region identifier component 114 may execute one or more portions of methods/processes 1200 and/or 1300 described below in connection with FIGS. 12 and 13, respectively. Further in some examples, the reconstruction generator component 116 may execute one or more portions of methods/processes 1100 and/or 1200 described below in connection with FIGS. 11 and 12, respectively.

In some examples, computing device 102 and/or server 104 can be any suitable computing device or combination of devices, such as a desktop computer, a mobile computing device (e.g., a laptop computer, a smartphone, a tablet computer, a wearable computer, etc.), a server computer, a virtual machine being executed by a physical computing device, a web server, etc. Further, in some examples, there may be a plurality of computing devices 102 and/or a plurality of servers 104.

In some examples, MRI data source 106 can be any suitable source of MRI data (e.g., data generated from an MRI machine). In a more particular example, MRI data source 106 can include memory storing MRI data (e.g., local memory of computing device 102, local memory of server 104, cloud storage, portable memory connected to computing device 102, portable memory connected to server 104, etc.). In another more particular example, MRI data source 106 can include an application configured to generate MRI data.

The MRI data 110 may correspond to a set of data samples that will be recognized by those of ordinary skill in the art as corresponding to magnetic resonance imaging techniques. For example, the MRI data may be imaging data that is generated by an MRI machine, such as when the MRI machine is being used to image an object. In some examples, MRI data source 106 can be local to computing device 102. Additionally, or alternatively, MRI data source 106 can be remote from computing device 102 and can communicate MRI data 110 to computing device 102 (and/or server 104) via a communication network (e.g., communication network 108).

In some examples, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard), a wired network, etc. In some examples, communication network 108 can be a local area network (LAN), a wide area network (WAN), a public network (e.g., the Internet), a private or semi-private network (e.g., a corporate or university intranet), any other suitable type of network, or any suitable combination of networks. Communication links (arrows) shown in FIG. 1 can each be any suitable communications link or combination of communication links, such as wired links, fiber optics links, Wi-Fi links, Bluetooth links, cellular links, etc.

An example technique for mitigating motion artifacts with MRI is to scan so quickly that there is little opportunity for motion during the scan time. With MRI, the scan time is proportional to the amount of data collected. Thus, one of ordinary skill will recognize the advantage to reconstruct images of high quality with little data collected. Existing technologies for doing so include partial Fourier sampling, parallel imaging, and compressed sensing. These techniques can be synergistically combined (e.g., in combination with aspects described herein) for even faster scanning than any one technique on its own.

Mechanisms described herein provide a new method of MRI acceleration that takes advantage of the known support of the image. While other acceleration methods can lead to image reconstruction algorithms that are computationally expensive (e.g., lengthening a time until an image is created), mechanisms provided herein present a relatively small computational burden. Furthermore, methods provided herein can be combined with existing techniques for even faster imaging.

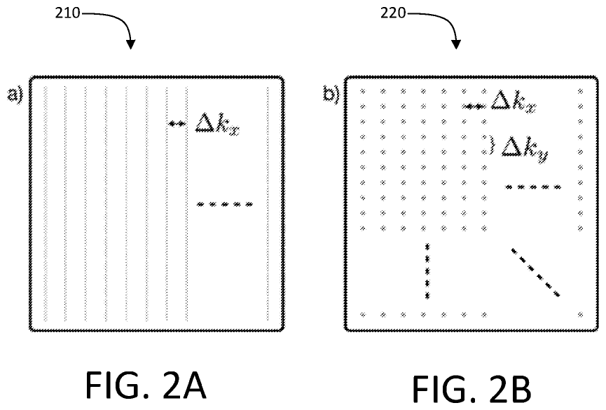

FIG. 2A illustrates a first sampling pattern 210 that takes into account a field-of-view and a field-of-relevance for a two-dimensional acquisition, and FIG. 2B illustrates a second sampling pattern 220 that takes into account a field-of-view and a field-of-relevance for a three-dimensional acquisition, according to some aspects described herein. The first sampling pattern 210 is a standard spin-warp (also known as 2DFT) acquisition. In FIG. 2A, points are sampled along each line. The first sampling pattern 210 is a two-dimensional acquisition and may be combined with a type of selective excitation to image (e.g., only image) a thin slice, which would produce a two-dimensional image. The first sampling pattern may be combined with a multi-slice excitation for simultaneous imaging of multiple slices. In FIG. 2B, each point represents a line coming out of the page, and points are sampled along the line. In that way, a three-dimensional dataset is sampled that can be used to reconstruct a three-dimensional image.

The variable Δkₓ of the first sampling pattern 210 and the second sampling pattern 220 defines a spacing between acquisitions in a first or x-direction. With a field-of-relevance, the variable Δkₓ can be made larger, which reduces the total scan time (since fewer scan lines are needed). Further, the variable Aky of the second sampling pattern 220 defines a spacing between acquisitions in a second or y-direction. With a field-of-relevance the variable Δkₓ and the variable Aky can be made larger, which reduces a total number of scan lines required, thereby reducing a total scan time. In the context of FIG. 2B, each point in the sampling pattern 220 represents a line coming out of the page (e.g., in the z-direction). Further, the spacing between samples, indicated with A symbols, may be inversely proportional to the corresponding length of the FOV. If A is smaller than this amount, then aliasing occurs.

During an MRI scanning protocol, three localizer or scout images may be acquired prior to imaging: one in an axial plane, one in a sagittal plane, and one in a coronal plane. It will be appreciated that fewer or additional localizer or scout images may be acquired in other examples. These images can permit a technician to verify that the patient is properly positioned in the scanner (with the relevant anatomy near the center of the machine's bore) and select the field-of-view. Some examples provided herein include modifying this interface to allow a technician to draw a non-rectangular field-of-view as well as a field-of-reference.

The sampling pattern cannot simply be modified to resemble the field-of-view. Instead, some examples provided herein may rely on the Fourier convolution theorem and the Fourier shift theorem to reduce the sampling pattern in such a way that the non-rectangular field-of-view and the field-of-relevance can be taken into account without substantially impacting the quality of a reconstructed image. The Fourier convolution theorem states the following: point-wise multiplication the frequency domain corresponds to convolution in the space domain. The Fourier shift theorem states the following: point-wise multiplication by a linear phase ramp in a frequency domain corresponds to a shift in a space domain.

After sampling in the Fourier domain, the resulting image is a sum of shifted versions of itself. If samples are sufficiently close together, then the shifted copies may lie outside of the field-of-view, and therefore are not seen by an end user in a reconstruction. This is a direct consequence of the convolution theorem: evenly-spaced sampling can be considered a multiplication by a comb function, and its inverse Fourier transform is also a comb function, where the spacing of the tines in the space domain is the inverse of the spacing in the frequency domain. The proposed technique, applicable with two-dimensional acquisitions (e.g., the first sampling pattern 210) or three-dimensional acquisitions (e.g., the second sampling pattern 220), assumes that data is collected on a Cartesian grid. For a two-dimensional acquisition, this would mean that equally spaced lines of data would be collected (one dimension of phase-encodings and one dimension of readout), as shown in the first sampling pattern 210. For a three-dimensional acquisition, this would mean lines are spaced in horizontal and vertical directions, as shown in the second sampling pattern 220. While the proposed technique is discussed above with respect to equally spaced, the proposed technique can be generalized to sampling that is not restricted to a Cartesian grid. For example, mechanisms could restrict distances in a non-Cartesian sampling based on the field-of-view.

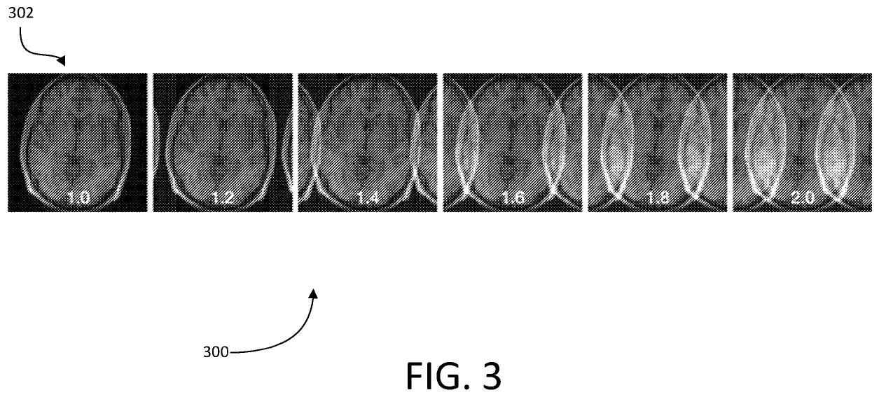

FIG. 3 illustrates a simulated magnetic resonance imaging reconstruction 300 of a brain, according to some aspects described herein. The reconstruction 300 includes a plurality of images 302 that each correspond to a different degree of sample spacing (e.g., 1.0, 1.2, 1.4, 1.6, 1.8, and 2.0). It should be understood that a sample spacing of 1.0 correspond to a fully sampled reconstruction (where the distances between samples correspond to the inverses of the lengths of the FOV), whereas a sample spacing of 2.0 corresponds to an under-sampled reconstruction with half as many samples. An under-sampled reconstruction is an image in which aliasing occurs across at least a portion of the image, as is discussed further herein.

The reconstruction 300 is generated based on two-dimensional simulated data, using the first sampling pattern 210 (see FIG. 2A), where columns of samples are separated in the Fourier domain. On the MRI machine, these images may be generated from a commonly used spin-warp (or 2DFT) acquisition, where a horizontal direction is a phase encode direction and a vertical direction is a readout direction. Alternatively, the horizontal direction could be a readout direction and the vertical direction could be the phase encode direction. The plurality of images 302 show how the image reconstructions 300 change when the spacing between sampled columns in the frequency domain (Δkₓ) is increased. As the spacing Δkₓ is increased, aliased copies of the image encroach into the FOV from both sides (e.g., as shown most clearly at a spacing of 2.0, relative to the fully sampled image at a spacing of 1.0). One should note that the center region of each of the plurality of images 302 remains uncorrupted (e.g., without aliasing) until the sample spacing exceeds a factor of 2. As such, mechanisms provided herein may use this property to reduce the number of samples while still yielding an uncorrupted reconstruction for a specified field-of-relevance.

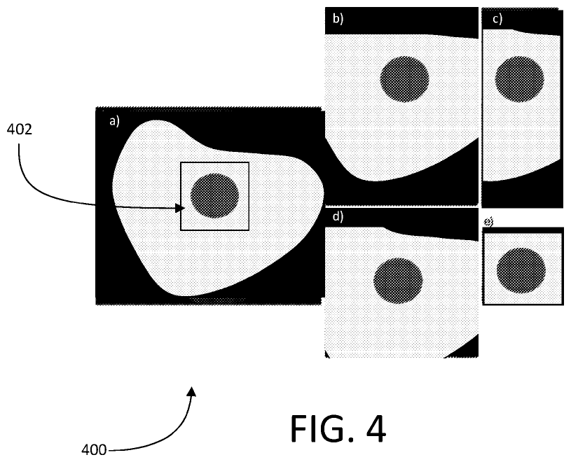

FIG. 4 illustrates a hypothetical abdominal magnetic resonance imaging (MRI) reconstruction 400 during pregnancy where the light region represents the torso and the embedded dark circle represents an axial slice of a fetal brain, according to some aspects described herein. The entire rectangular image of frame “a” represents the field-of-view. A field-of-relevance 402, drawn on the image of frame “a”, may be provided by an MRI scanning technologist or other medical professional or, as another example, may be automatically determined. Note that there could be significant aliasing with the image 400 encroaching in from the sides without affecting the field-of-relevance 402. Mechanisms provided herein may thus intentionally allow such aliasing to occur. By allowing aliasing to intentionally occur, outside of a field-of-relevance, scan times are able to be significantly reduced, using mechanisms described herein.

In some examples, aspects described herein include generating and/or displaying a user-interface (UI), such as a graphical user-interface (UI). In some examples, the GUI may be displayed via a display of a computing device, such as the computing device 102 of FIG. 1. In some examples, an indication corresponding to a field-of-relevance (e.g., the field-of-relevance 402) and/or a field-of-view may be received via the GUI. In some examples, the indication includes a generally non-rectangular shape. For example, the generally non-rectangular shape can be at least one of a shape selected from a library of shapes, a drawn shape, and/or a system-generated shape.

In some examples, a technologist (or other user) may select the generally non-rectangular shape from a library of shapes that includes an oval, circle, triangle, knee shape, shoulder shape, foot shape, or another generally non-rectangular shape which may be recognized by those of ordinary skill in the art. In some examples, a technologist may draw (e.g., free hand, such as via a stylus, mouse, gaze input, touchpad, etc.) the generally non-rectangular shape. In some examples, a system can include a model, such as a machine-learning (e.g., deep-learning) model which is trained to automatically and/or in-response to a prompt, generate the generally non-rectangular shape. In some examples, the system-generated shape may be a recommended shape, which a user can select, modify, and/or replace with their own selected and/or drawn shape.

In some examples, a plurality of indications may be received via the GUI, such as a first indication corresponding to the field-of-relevance 402 and/or a field-of-view and a second indication corresponding to a field-of-view and/or the field-of-relevance 402. In some examples, each of the indications includes a generally-nonrectangular shape generated according to mechanisms described herein.

Frame “a” of FIG. 4 shows a fully sampled reconstruction; the rectangular contour, which encloses the fetal brain, is the field-of-relevance 402. Frames “b” and “c” show simulations of reconstruction after separating columns in a two-dimensional acquisition before and after cropping of aliased portions, respectively. Frames “d” and “e” show simulations of reconstructions after separating samples in a three-dimensional acquisition before and after cropping of the aliased portions, respectively. In frames “c” and “e,” relevant portion remains uncorrupted by aliasing for analysis (e.g., by a clinician).

A sampling pattern to be created, using mechanisms provided herein, uses a difference between the field-of-view and the field-of-relevance (e.g., field-of-relevance 402) to determine a maximum spacing that can be used for imaging without permitting aliasing to encroach on the field-of-relevance. For example, in the case where mechanisms provided herein are performing two-dimensional imaging according to the sampling pattern of FIG. 2A, Δkₓ can be:

$$Δ {k}_{x} = \frac{1}{{FOR}_{w} + max ( {FOV}_{w} - {FOR}_{R} , {FOR}_{L} )}$$

where FOR_{w} and FOV_{w} are the widths of the field-of-relevance and the field-of-view, respectively; and FOR_{L} and FOR_{R} are the left and right coordinates of the field-of-relevance, respectively. This spacing permits aliasing outside of the field-of-relevance 402, but keeps the region inside of it free of artifacts.

Some examples provided herein include creating an image from a sampling pattern by: 1) multiplying scan data by a linear ramp, so that the center of the field-of-relevance is located in the center of the field-of-view (in accordance with the Fourier shift theorem), 2) perform an inverse fast Fourier transform (FFT), and 3) crop the aliased portion and retain an uncorrupted image of the field-of-relevance. In some examples, the FFT may be a non-uniform FFT. Alternatively, in some examples, the FFT may be a uniform FFT. As noted above, an example of this process is shown in FIG. 4, where frame “b” of FIG. 4 shows the result after the inverse FFT of step 2 and frame “c” of FIG. 4 shows the result after the cropping of step 3. Note that the field-of-relevance 402 is presented without aliasing. With this approach, the number of samples has been reduced by 30%, which may equate to a 30% reduction in scan time.

With three-dimensional imaging using the sampling pattern 220 (FIG. 2B), the horizontal spacing between readout lines Δkₓ can be set as before, and Δk_{y} can be set according to

$$Δ {k}_{y} = \frac{1}{{FOR}_{h} + max ( {FOV}_{h} - {FOR}_{T} , {FOR}_{B} )}$$

where FORₕ and FOVₕ are the heights of the field-of-relevance and the field-of-view, respectively; and FOR_{B} and FOR_{T} are the bottom and top coordinates of the field-of-relevance, respectively. This spacing permits aliasing in two dimensions outside of the field-of-relevance 402, but keeps the region inside of it free of artifacts.

Frame “d” of FIG. 4 shows a result after performing an inverse FFT. Frame “e” shows a result after cropping out a field-of-relevance, which is free of any aliasing. In this case, the number of samples has been reduced by 50%, which could be scanned in half the time.

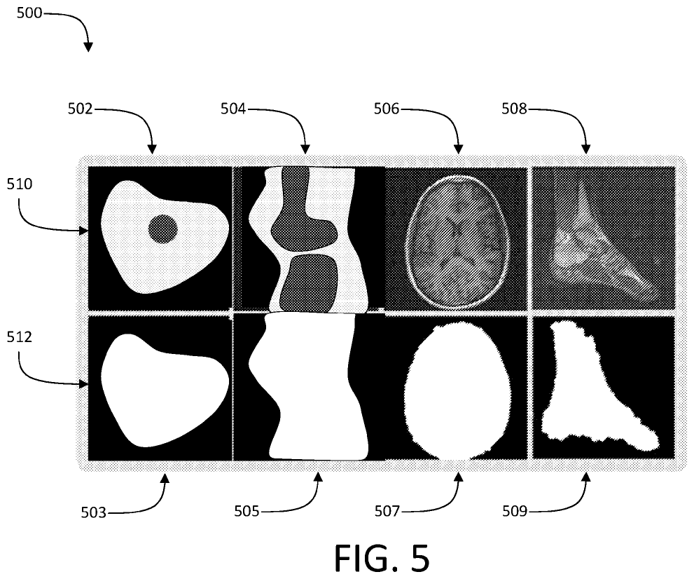

FIG. 5 illustrates a magnetic resonance imaging (MRI) reconstruction 500 of a plurality of components of a body, such as a pregnant woman 502, a knee 504, a brain 506, and an ankle 508, according to some aspects described herein. The MRI reconstruction 500 includes a top row 510 that shows the pregnant woman 502, the knee 504, the brain 506, and the ankle 508, and a bottom row 512 that shows a first estimate of the support 503, a second estimate of the support 505, a third estimate of the support 507, and a fourth estimate of the support 509 that correspond to each of the pregnant woman 502, the knee 504, the brain 506, and the ankle 508, respectively.

Conventional MRI accurately reconstructs an entire rectangular field-of-view, which is supplied by a scanning technologist or other person who controls the MRI machine for scanning (e.g., a researcher or a scientist). To date, because of technical limitations, the field-of-view has always been a rectangle that encompasses a subject to be imaged. The subject is rarely a rectangle; thus, to avoid aliasing, the field-of-view in conventional MRI contains a region outside of the subject which appears black. Examples of this are shown in the top row of FIG. 5 (e.g., pregnant woman 502, knee 504, brain 506, and ankle 508). Further, in some examples, the rectangle may be rotated. Namely, the field-of-view may still be a rectangle, but it might not have edges that are aligned with any pre-defined coordinate system and it may not be aligned with the edges of localizer images.

It is desirable for a scanning technologist to be able to provide a non-rectangular field-of-view that excludes the regions where there is not any subject to be imaged, as such information may lead to a faster scan for a given resolution. The non-rectangular field-of-views are shown in the bottom row of FIG. 5 (e.g., the first field-of-relevance 503, the second field-of-relevance 505, the third field-of-relevance 507, and the fourth field-of-relevance 509). Since MRI is a Fourier sensing machine (where data is collected in the frequency domain), it has not previously been known how to take advantage of a non-rectangular field-of-view. However, mechanisms provided herein satisfy such a need.

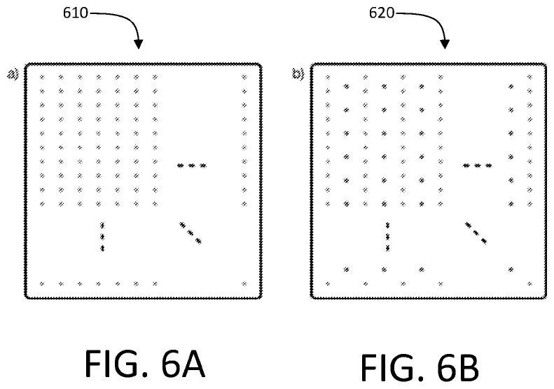

FIG. 6A illustrates a first sampling pattern 610 that may be generated using conventional techniques, that take a full width and height of a rectangular field-of-view into account, whereas FIG. 6B illustrates a second sampling pattern 620 that can be used to take a non-rectangular field-of-view into account, according to some aspects described herein.

Mechanisms provided herein are applicable to three-dimensional MRI where parallel lines of data are collected. For example, in the first sampling pattern 610 and the second sampling pattern 620, each point in the sampling patterns 610, 620 represents a line of data coming out of the page. With an acquisition of parallel lines, processing can take place for each slice independently (e.g., by inverse Fourier transforming the data along the dimension of the readout lines and placing the data in a kₓ, k_{y}, z hybrid space). While examples provided herein may be discussed with respect to two-dimensional slices, it should be recognized by those of ordinary skill in the art that the same or similar methods and/or systems may be applied for all slices in a volume that is to be imaged, thereby applying mechanisms described herein to three-dimensional imaging. Further, mechanisms described herein could be used for types of MRI imaging where lines of data are collected that are not parallel and/or where data does not lie on a Cartesian grid. In such cases, sampling lines would be chosen so that distances between samples would be bounded based on expressions presented. Alternatively, in some examples, the data can be retained in the Fourier domain and processed as a three-dimensional volume. In such examples, the non-rectangular field-of-view can be taken into account in two or three of the dimensions of the volume as described below.

The first sampling pattern 610 takes into account a full width and height of a field-of-view. The second sampling pattern 620 takes a non-rectangular field-of-view into account. Note that the total number of samples is reduced in the second sampling pattern 620. Since the scan time of MRI is proportional to the sampling pattern, using the second sampling pattern 620 translates into a faster scan for an image of the same resolution.

In some examples with two-dimensional imaging, non-Cartesian sampling can be reconstructed by interpolating points on the second sampling pattern shown in 620 and reconstructing the image as described herein.

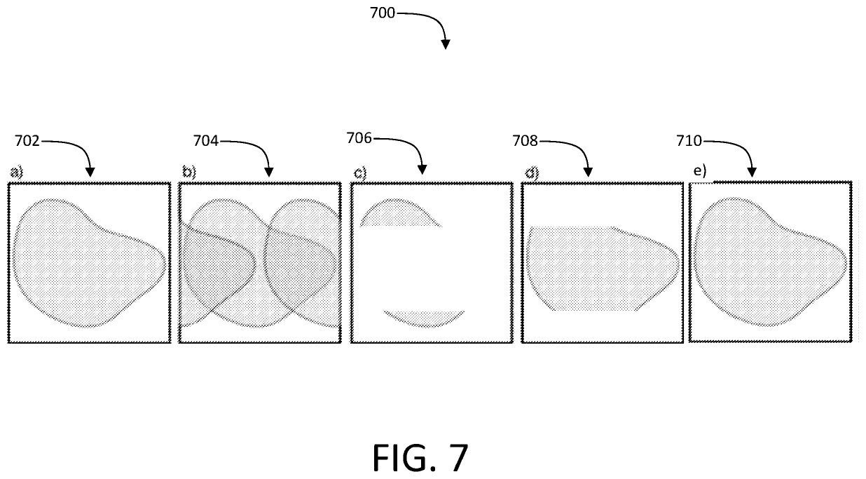

FIG. 7 illustrates a depiction of a flow 700 to reconstruct an image 702 with a non-rectangular field-of-view, from fewer samples, according to some aspects described herein. Initially, the non-rectangular field-of-view is shown in 702. Image 704 is a reconstruction with an inverse two-dimensional FFT only using gray sample points of FIG. 6B. The gray sample points have a vertical spacing equal to the inverse of the total vertical extent of the image, and have a horizontal spacing equal to double the inverse of the total horizontal extent of the image.

It should be recognized that while the example flow 700 of FIG. 7 includes a horizontal spacing equal to double (or a factor of 2) the inverse of the total horizontal extent of the image, any spacing greater than or equal to 1 can be used. A spacing of 1 may be the fully sampled FOV sampling pattern of FIG. 6A. If a spacing other than 2 were used, then modifications may be made to the mechanisms described herein, as will be recognized by those of ordinary skill in the art. For example, there may be aliasing with copies of the image encroaching in from the sides, as shown in FIG. 3, but a degree of the aliasing may be less than when a factor of 2 was used. In some examples, the number of rows containing data that are uncorrupted would be different. Generally, sampling patterns may be used for many different factors (e.g., 1.1, 1.2, 1.3, . . . , 3.8, 3.9, 4.0, etc.), for example, to see which sampling pattern produces the fewest number of samples. In some examples, the sampling pattern of 220 may be rotated for each factor and evaluated as to whether or not rotating reduces the number of samples required. In some examples, the sampling pattern that produces the fewest number of samples may be used for reconstruction.

When compared to the fully sampled pattern of FIG. 6A, it is evident that alternating columns of data have not been included with the gray points of the sample pattern of FIG. 6B. Because of this omission of data columns, a reconstructed image 704 is the sum of the image 702 with a copy of itself shifted by half the horizontal field-of-view (thus introducing aliasing into the reconstruction with support depicted in image 704). Isolating those rows without any overlap and retaining the region that is within the non-rectangular field-of-view yields a subtracted image with support shown in image 706. That is, even though there was aliasing, the non-rectangular field-of-view allowed for reconstructing outer regions without any artifacts.

Taking the Fourier transform of the image corresponding to support shown in 706 and subtracting it away from the collected samples yields values from the Fourier transform of an image with support shown in 708. Note that the vertical extent of the Fourier transformed image 708 is less than that of the initial image 702. The vertical spacing between the blue alternating columns of FIG. 6B is equal to the inverse of this reduced vertical extent. The image with reduced vertical extent is reconstructed by performing an inverse Fourier transform on the subtracted Fourier values. By summing together the image of 706 and the image of 708, a final accurate image with support 710 can be reconstructed.

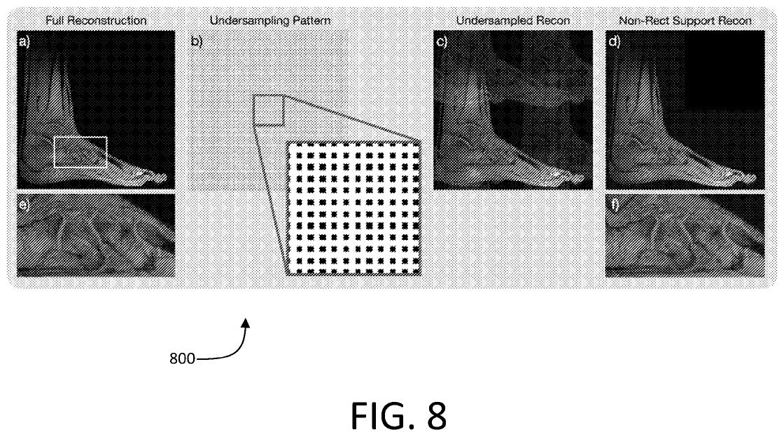

FIG. 8 illustrates a depiction 800 of reconstructions of an ankle. The data was collected from an 8-coil parallel image acquisition using a 3 Tesla General Electric Healthcare MRI machine. Frame “a” shows a fully sampled reconstruction. Frame “e” is the portion outlined in white from Frame “a” and is enlarged to better observe fine details of the enlarged portion of the fully sampled reconstruction. Frame “b” is a reduced sampling pattern comprised only of every other row and every other column of the original fully sampled pattern. Frame “c” is the zero-filled reconstruction, where uncollected values are assumed to be 0. That is, black spots in frame “b” are considered to be 0. It is noted that the shifted aliasing ghosts of the image are imprinted into the image itself. Frame “d” shows the reconstruction from the reduced sampling pattern while accounting for the non-rectangular field-of-view. It is noted that the aliasing corruptions have been eliminated and that there is not any noticeable difference between frame “d” and frame “a”. Frame “f” is the portion of frame “d” corresponding to the white rectangle in frame “a” and has been enlarged. It is noted that there is not any noticeable difference between frame “f” and frame “e”.

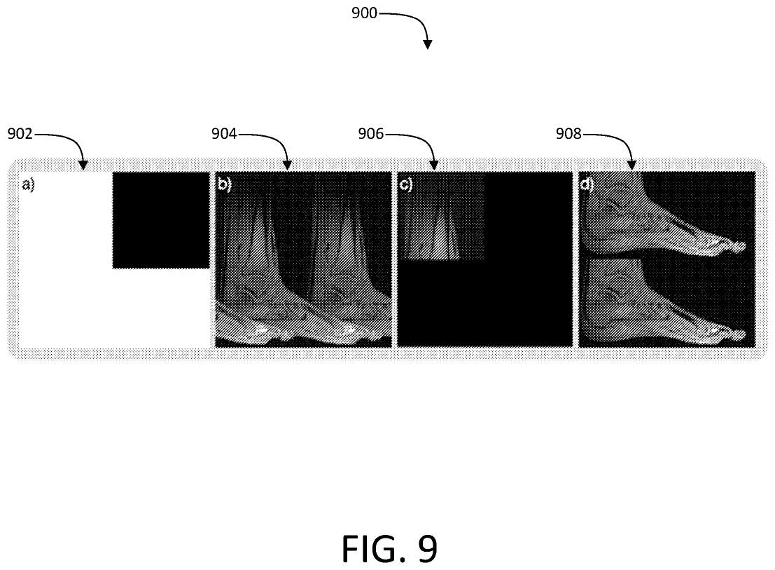

FIG. 9 illustrates a depiction 900 of generating a full reconstruction, while accounting for a non-rectangular field-of-view, according to some aspects described herein. Frame “a” shows a specified support 902, as the white region of frame “a.” The non-rectangular field-of-view would be the boundary of the white region of frame “a.” Frame “b” shows a first reconstruction 904 from columns of k-space data. Frame “c” shows a top-half portion of frame “b” where values not in the support have been set to 0 and appear black. Frame “d” shows a second reconstruction 908 from rows of k-space data after subtracting the Fourier transform of frame “c.” A full reconstruction may thus be created by summing together what is illustrated in frame “c” with the bottom half of what is illustrated in frame “d,” thereby yielding a full MRI reconstruction of the imaged ankle in the support region (e.g., as indicated by frame “a”).

Generally, FIG. 9 illustrates a non-rectangular field-of-view as well as intermediate results of a reconstruction algorithm, according to aspects described herein. By taking a non-rectangular field-of-view into account, the number of samples required to reconstruct the image has been reduced by about 25% in the depiction 900 of generating a full reconstruction.

An immediate gain can be obtained by combining the two acceleration approaches herein, which include permitting a user (e.g., a scanning technologist) to provide a non-rectangular field-of-view, as well as to provide a non-rectangular field-of-relevance. The combination would permit additional spacing between samples in a sampling pattern and further accelerate an MRI acquisition.

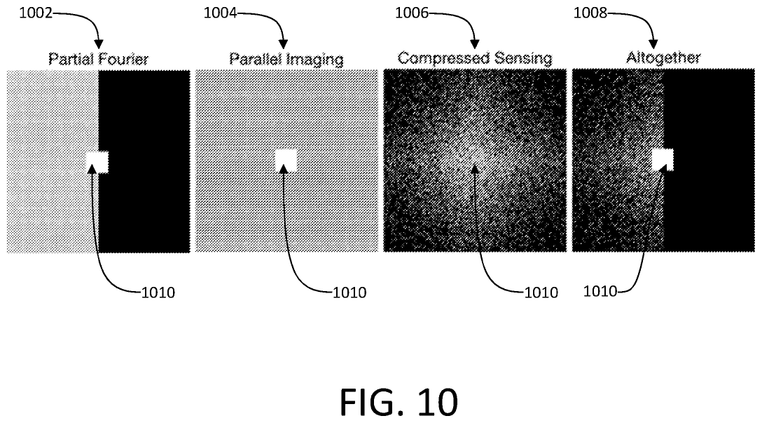

FIG. 10 illustrates a plurality of sampling patterns, according to some aspects described herein, including a partial Fourier sampling pattern 1002, a parallel imaging sampling pattern 1004, a compressed sensing sampling pattern 1006, and an altogether sampling pattern 1008.

The mechanisms described herein can be combined with existing acceleration methods, including, for example, the partial Fourier sampling 1002, the parallel imaging 1004, and the compressed sensing 1006. For each of these techniques, a fully sampled small region 1010 centered on 0 frequency may be collected. The sampling patterns for the combination algorithms with three-dimensional imaging are presented in FIG. 10. The fully sampled region 1010 is a white rectangle in the center of each pattern. With knowledge of the non-rectangular field-of-view and non-rectangular field-of-relevance, mechanisms provided herein are able to reduce a number of samples of this fully sampled region 1010, as shown in FIG. 6B.

Partial Fourier sampling collects data from a little more than half of a Fourier domain. The phase of the final collected data is estimated using the fully sampled center region 1010, this phase is removed, and then the image is assumed to be real to fill in missing data. With knowledge of a non-rectangular support, samples from the half of the Fourier domain that is normally collected can be eliminated while still accurately reconstructing the image, as shown in the partial Fourier sampling pattern 1002.

The combination of mechanisms disclosed herein with parallel imaging can reduce a length of a scan. Parallel imaging uses multiple coils that can each image the body from a different vantage point. Each coil simultaneously collects data during the MRI acquisition with a unique sensitivity region. Therefore, rather than having a scanning technician provide a field-of-view of an entire image, the scanning technician can provide a non-rectangular field-of-view for each coil. Or, the field-of-view for each coil can be automatically determined, at least in part. Due to the reduced extent, the samples in the Fourier domain can be further separated, thereby reducing the total number of samples required to achieve a given resolution. This can be combined with the assumption of linear predictability to eliminate complete rows from a remaining set of collected data, as shown in the parallel imaging sampling pattern 1004. Alternatively, if the sensitivity of each coil is known, this can be combined with a reduced sampling pattern where nearby points in the Fourier domain are interpolated with a model-based reconstruction or SENSE algorithm.

Compressed sensing (e.g., compressed sensing 1006) utilizes a variable density sampling pattern and the assumption of sparsity in a transformed domain (e.g., the wavelet domain) in order to accurately reconstruct an image. The image is reconstructed by solving an optimization problem, which can be solved numerically. The sampling pattern that combines compressed sensing with a non-rectangular field-of-view and a non-rectangular field-of-relevance is shown in 1006. This can be further combined with parallel imaging using a model-based reconstruction, where the model includes multiplication by the sensitivities of each individual coil.

The sampling pattern that combines all acceleration methods is shown in the altogether sampling pattern 1008. As with standard compressed sensing, the reconstructed image is the result of an optimization problem. Intuitively, an image is reconstructed in steps: use the assumption of sparsity to fill in enough data for parallel imaging; use the assumption of linear predictability to fill in missing rows; use the assumption of slowly varying phase to fill in the missing half of k-space; and then use knowledge of the non-rectangular support and the non-rectangular field-of-relevance to interpolate any remaining missing data. For example, this can be accomplished by solving the following optimization problem:

$${minimize}_{x} {‖ Ψ x ‖}_{1} subject to {‖ ℳ 𝒢 {𝒟}_{𝒫 , 𝒮} FSx - ℋ b ‖}_{2} < ϵ and {‖ x ( {𝒮}^{𝒞} ) ‖}_{2} < ϵ$$

In the above example, b is the data vector collected, [symbol] is the transformation that performs correction for partial Fourier sampling with Homodyne detection, x is the optimization variable that represents the image to be reconstructed, S represents a multiplication by the coils' sensitivity maps, F is the Discrete Fourier Transform, [symbol] is a binary mask that isolates those sample points required when parallel imaging and undersampling with the support are combined, [symbol] is the linear transform that interpolates missing points with the GRAPPA kernel, [symbol] is the sampling mask, ϵ is a bound on the noise magnitude, and x( [symbol] ) are those voxels of the image that lie outside of (in the complement of) the support.

In some examples, the processing that takes a non-rectangular field-of-view and/or a non-rectangular field-of-relevance may be included as one or more layers in a neural network. In some examples, the generating of one or more reconstructions includes processing by one or more layers in a neural network.

FIG. 11 illustrates an overview of an example method to reconstruct an image, from an MRI machine, with reduced sampling, according to some aspects described herein. In accordance with some examples, aspects of method 1100 are performed by a device, such as computing device 102 and/or server 104 discussed above with respect to FIG. 1.

Method 1100 begins at operation 1102, where a first indication is received that corresponds to a support region. In some examples, the support region is generally rectangular. Alternatively, in some examples, the support region is generally non-rectangular. For example, referring back to the MRI reconstruction 500 of FIG. 5, a plurality of subjects are illustrated that include support regions that are non-rectangular (e.g., corresponding to the pregnant woman 502, knee 504, brain 506, and ankle 508).

At operation 1104, a set of data samples associated with the first indication are received. In some examples, the set of data samples associated with the first indication are obtained (e.g., from MRI data source 106). The set of data samples include one or more regions having aliasing. The set of data samples may be received from an imaging device. The imaging device may be similar to the MRI data source 106 described earlier herein with respect to FIG. 1. For example, the imaging device may be an MRI machine.

In some examples, the set of data samples are received from an under-sampled reconstruction. The under-sampled reconstruction may be a sample in which aliasing is present across at least a portion of a field-of-view defined by the set of data samples. For example, as shown FIG. 7, the reconstructed image 704 is the sum of the image 702 with a copy of itself shifted by half of a horizontal field-of-view, such that aliasing is present across half of the field-of-view. As another example, FIG. 3 illustrates the reconstruction 300, where depending on a spacing of an interval at which an object is imaged, a related amount of aliasing is generated.

At operation 1110, a subset of data samples, from the set of data samples, is determined. The subset of data samples can be determined based on the support region. Additionally, or alternatively, in a three-dimensional acquisition, for example, a plurality of different Δkₓ and Δk_{y} may be tested and a pair of Δkₓ and Δk_{y} values may be chosen that yield the smallest number of samples. The subset of data samples may be determined based on the known values of Δkₓ and Δk_{y} specified using mechanisms described herein. The subset of data samples may correspond to a reconstruction that does not overlap with the one or more regions having aliasing (e.g., as shown in the subtracted image 706 of FIG. 7).

At operation 1112, a first reconstruction is generated, based on the subset of data samples. As an example, the subtracted image 706 may be a first reconstruction that is generated based on the subset of data samples that generated the illustrations of FIG. 7.

At operation 1114, a frequency representation that corresponds to the first reconstruction is generated. The frequency representation may correspond to a plurality of data points or signals in the frequency domain. It is noted that the set of data samples are also in the frequency domain. The frequency representation may be generated based on a Fourier transform, such as a uniform and/or non-uniform Fourier transform technique described earlier herein.

At operation 1116, the frequency representation is subtracted from the set of data samples, thereby generating a modified first set of data samples. For example, the set of data samples may be stored in a database, or a repository, or another form of memory, from which the frequency representation is subtracted.

At operation 1118, a second reconstruction is generated, based on the modified set of data samples. As an example, the second reconstruction may be similar to the Fourier transformed image 708 of FIG. 7. For instance, the Fourier transformed image 708 corresponds to the image 702 after the subset of data points in the subtracted image 706 are removed from the data points in the image 702. Additional and/or alternative examples will be recognized by those of ordinary skill in the art, at least in light of teachings described herein.

At operation 1120, a reduced sampling reconstruction is generated, by combining the first and second reconstructions (e.g., as were generated at operations 1112 and 1118, respectively). In some examples, the first and second reconstructions are generated using an inverse two-dimensional fast Fourier transform (FFT). In some examples, the FFT may be uniform, whereas in some examples, the FFT may be non-uniform. Additional and/or alternative techniques for generating the first and second reconstructions may be recognized by those of ordinary skill in the art.

At operation 1122, the reduced sampling reconstruction is output. For example, the reduced sampling reconstruction may be output to the computing device 102. Alternatively, the reduced sampling reconstruction may be output to the server 104. The reduced sampling reconstruction may be further processed by the computing device 102, the server 104, and/or another device that may be recognized by those of ordinary skill in the art.

Method 1100 may terminate at operation 1122. Alternatively, method 1100 may return to operation 1102, from operation 1122, to provide a continuous feedback loop of receiving a first indication corresponding a support region and a set of data samples that includes aliasing, to generate and output a reduced sampling reconstruction.

FIG. 12 illustrates an overview of an example method to reconstruct an image, from an MRI machine, with reduced sampling, according to some aspects described herein. In accordance with some examples, aspects of method 1200 are performed by a device, such as computing device 102 and/or server 104 discussed above with respect to FIG. 1.

Method 1200 beings at operation 1202, where an indication is received that corresponds to a field-of-relevance. In some examples, a total field of view (e.g., that encompasses an entire patient) is also received. In some examples, the indication that corresponds to the field-of-relevance is received via user-input. For example, a technician of an MRI machine may circle the field-of-relevance (e.g., on a computing device) to indicate where the field-of-relevance is located. Additionally, or alternatively, in some examples, the indication corresponding to the field-of-relevance is received automatically. For example, the field-of-relevance may be determined via a sensor (e.g., a proximity sensor, a visual sensor, etc.) and/or recognized via a visual processing algorithm (e.g., an artificially intelligent and/or machine learning algorithm). The field-of-relevance may correspond to an object that is desired to be imaged (e.g., a kidney, a fetal brain, a prostate, etc.).

At operation 1204, a sampling interval is generated, based on the received indication, causing the MRI machine to collect data samples at the sampling interval. In some examples the sampling interval is a distance interval. For example, the sampling interval may include a fixed interval of horizontal distances at which the data samples are received. Additionally, or alternatively, the sampling interval may include a fixed interval of vertical distances at which the data samples are received (e.g., as were discussed with respect to FIGS. 2A-2B). In some examples, when the sampling interval includes an interval in a first direction, the sampling interval may correspond to two-dimensional imaging (e.g., as shown in FIG. 2A). In some examples, when the sampling interval includes an interval in a first direction and a second direction, the sampling interval may correspond to three-dimensional imaging (e.g., as shown in FIG. 2B).

At operation 1206, a reconstruction is generated, based on the data samples collected from the sampling interval. As a result of the sampling interval generated at operation 1204, the reconstruction generated at operation 1206 may have aliasing. In some examples, the reconstruction is intentionally generated to have aliasing, which may be contrary to conventional techniques for MRI image reconstruction where aliasing may be viewed as undesirable. Examples of aliasing are shown and described herein, for example, with respect to FIGS. 3, 4, 7, and 9. By allowing aliasing to intentionally occur, outside of a field-of-relevance, scan times are able to be reduced, using mechanisms described herein.

At operation 1208, a region of the reconstruction that corresponds to the field-of-relevance is determined. The determined region does not have aliasing. In some examples, operation 1208 includes cropping the reconstruction (e.g., generated at operation 1206), to omit aliasing that occurs external to the field-of-relevance (e.g., based on the indication of operation 1202). Additionally or alternatively, in some examples, operation 1208 includes generating a subset of data, from a set of data corresponding to the reconstruction, that corresponds to the field of relevance. The subset of data corresponds to the region of the reconstruction that corresponds to the field of relevance.

At operation 1210, the region of the reconstruction that corresponds to the field-of-relevance is output. For example, the region may be output to the computing device 102. Alternatively, the region may be output to the server 104. The region may be further processed by the computing device 102, the server 104, and/or another device that may be recognized by those of ordinary skill in the art, for example using image processing techniques that may be recognized by those of ordinary skill in the art.

Method 1200 may terminate at operation 1210. Alternatively, method 1200 may return to operation 1202, from operation 1210, to provide a continuous feedback loop of receiving an indication corresponding to a field-of-relevance, generating a sampling interval, based on the received indication, that causes an MRI machine to collect data samples at the sampling interval, generating a reconstruction, and outputting a region of the reconstruction that corresponds to the field-of-relevance.

FIG. 13 illustrates a block diagram illustrating example physical components of a computing device with which aspects of the disclosure may be practiced. The device may be a mobile computing device, for example. One or more of the present embodiments may be implemented in an operating environment 1300. This is only one example of a suitable operating environment and is not intended to suggest any limitation as to the scope of use or functionality. Other well-known computing systems, environments, and/or configurations that may be suitable for use include, but are not limited to, personal computers, server computers, hand-held or laptop devices, multiprocessor systems, microprocessor-based systems, programmable consumer electronics such as smartphones, network PCs, minicomputers, mainframe computers, distributed computing environments that include any of the above systems or devices, and the like.

In its most basic configuration, the operating environment 1300 typically includes at least one processing unit 1302 and memory 1304. Depending on the exact configuration and type of computing device, memory 1304 (e.g., instructions for one or more aspects disclosed herein, such as one or more aspects of methods/processes 1100 and 1200, described with respect to FIGS. 11 and 12, respectively) may be volatile (such as RAM), non-volatile (such as ROM, flash memory, etc.), or some combination of the two. This most basic configuration is illustrated in FIG. 13 by dashed line 1306. Further, the operating environment 1300 may also include storage devices (removable, 1308, and/or non-removable, 1310) including, but not limited to, magnetic or optical disks or tape. Similarly, the operating environment 1300 may also have input device(s) 1314 such as remote controller, keyboard, mouse, pen, voice input, on-board sensors, etc. and/or output device(s) 1312 such as a display, speakers, printer, motors, etc. Also included in the environment may be one or more communication connections 1316, such as LAN, WAN, a near-field communications network, a cellular broadband network, point to point, etc.

Operating environment 1300 typically includes at least some form of computer readable media. Computer readable media can be any available media that can be accessed by the at least one processing unit 1302 or other devices comprising the operating environment. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other tangible, non-transitory medium which can be used to store the desired information. Computer storage media does not include communication media. Computer storage media does not include a carrier wave or other propagated or modulated data signal.

Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media.

The operating environment 1300 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet.

Aspects of the present disclosure, for example, are described above with reference to block diagrams and/or operational illustrations of methods, systems, and computer program products according to aspects of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

The description and illustration of one or more aspects provided in this application are not intended to limit or restrict the scope of the disclosure as claimed in any way. The aspects, examples, and details provided in this application are considered sufficient to convey possession and enable others to make and use claimed aspects of the disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

What is claimed is:

1. A method to reconstruct an image, from a magnetic resonance imaging (MRI) machine, with reduced sampling, the method comprising:
receiving a first indication corresponding to a support region;
obtaining a set of data samples associated with the first indication, the set of data samples comprising one or more regions having aliasing;
determining a subset of data samples, from the set of data samples, that does not overlap with the one or more regions having aliasing;
generating a first reconstruction, based on the determined subset of data samples;
generating a frequency representation corresponding to the first reconstruction;
subtracting the frequency representation from the set of data samples, thereby generating a modified set of data samples;
generating a second reconstruction, based on the modified set of data samples;
generating a reduced sampling reconstruction corresponding to the support region, by combining the first and second reconstructions; and
outputting the reduced sampling reconstruction.

2. The method of claim 1, wherein the set of data samples are from an under-sampled reconstruction, the under-sampled reconstruction being a sample in which aliasing is present across at least a portion of a field-of-view corresponding to the set of data samples.

3. The method of claim 2, wherein the at least a portion of the field-of-view is half of the field-of-view.

4. The method of claim 1, wherein the set of data samples are obtained from an imaging device.

5. The method of claim 1, wherein the support region is generally non-rectangular.

6. The method of claim 1, wherein the first and second reconstructions are generated using an inverse two-dimensional fast Fourier transform (FFT).

7. The method of claim 1, wherein the set of data samples are received from one or more sensing coils and a field-of-view is provided for each sensing coil of the one or more sensing coils.

8. The method of claim 1, wherein the generating of the first reconstruction and the generating of the second reconstruction comprise processing by one or more layers in a neural network.

9. A method to reconstruct an image, from a magnetic resonance imaging (MRI) machine, with reduced sampling, the method comprising:
receiving an indication corresponding to a field-of-relevance;
generating a sampling interval, based on the received indication, causing the MRI machine to collect data samples at the sampling interval; and
generating a reconstruction, based on the data samples collected for the sampling interval, wherein the reconstruction has aliasing resulting from the sampling interval.

10. The method of claim 9, further comprising:
determining a region of the reconstruction corresponding to the field-of-relevance, wherein the determined region does not have aliasing; and
outputting the region of the reconstruction.

11. The method of claim 9, wherein the sampling interval comprises a first fixed interval of distances in a first direction at which the data samples are obtained.

12. The method of claim 11, wherein the sampling interval further comprises a second fixed interval of distances in a second direction at which the data samples are obtained.

13. The method of claim 12, wherein the first direction is a horizonal direction and the second direction is a vertical direction.

14. The method of claim 9, wherein the indication corresponding to the field-of-relevance is received via user-input.

15. The method of claim 9, wherein the indication corresponding to the field-of-relevance is generated automatically.

16. The method of claim 9, wherein the indication corresponding to the field-of-relevance is generated automatically based on a localizer scan obtained from the MRI machine.

17. The method of claim 9, wherein the generating of the reconstruction comprises processing by one or more layers in a neural network.

18. A method to reconstruct an image, from a magnetic resonance imaging (MRI) machine, the method comprising:
displaying a graphical user-interface (GUI);
receiving, via the GUI, an indication corresponding to one of a field-of-relevance or a field-of-view, wherein the indication comprises a generally non-rectangular shape;
generating a sampling interval, based on the received indication; and
causing the MRI machine to collect data samples at the sampling interval corresponding to the generally non-rectangular shape.

19. The method of claim 18, wherein the indication is a first indication, and wherein the method further comprises receiving a second indication corresponding to the other one of a field-of-relevance or a field-of-view.

20. The method of claim 18, wherein the generally non-rectangular shape is at least one of:
a user selection of the non-rectangular shape from a library of shapes;
a shape drawn by the user via the GUI; or
a system-generated shape.
